(19) United States
(12) Patent Application Publication
(10) Pub. No.: US 2024/0148309 A1
(43) Pub. Date: May 9, 2024

(54) CHRONIC PERIODIC MONITORING FOR ATRIAL TACHYARRHYTHMIA DETECTION

(71) Applicant: Cardiac Pacemakers, Inc., St. Paul, MN (US)

(72) Inventors: David L. Perschbacher, Blaine, MN (US); Allan T. Koshiol, Lino Lakes, MN (US); Deepa Mahajan, North Oaks, MN (US); Sunipa Saha, Shoreview, NC (US)

(21) Appl. No.: 18/385,208

(22) Filed: Oct. 30, 2023

Related U.S. Application Data
(60) Provisional application No. 63/422,320, filed on Nov. 3, 2022.

(51) Int. Cl. A61B 5/364; A61B 5/00; A61B 5/361

(52) U.S. Cl. CPC A61B 5/364; A61B 5/0002; A61B 5/361; A61B 5/7221; A61B 5/7275; A61B 5/7282; A61B 5/742; A61B 2560/02

(57) ABSTRACT

Systems and methods for detecting cardiac arrhythmia are discussed. A medical-device system includes an ambulatory monitor device and an arrhythmia analysis device communicatively coupled to each other. The ambulatory monitor device can sense a cardiac signal from the patient, and intermittently collect data segments of the cardiac signal in accordance with a data segment collection rate or schedule over a monitoring period. The data segments each has a specific segment duration. The monitor device can transmit the intermittently collected data segments to the arrhythmia analysis device in accordance with a transmission schedule or in response to a trigger event. The arrhythmia analysis device can detect a cardiac arrhythmia from the intermittently collected data segments. The segment duration or the data segment collection rate or schedule may be adjusted based on a performance measure of the arrhythmia detection.

### CLAIM OF PRIORITY

This application claims the benefit of U.S. Provisional Application No. 63/422,320, filed on Nov. 3, 2022, which is hereby incorporated by reference in its entirety.

### TECHNICAL FIELD

This document relates generally to medical devices, and more particularly, to systems, devices, and methods for detecting atrial tachyarrhythmia.

### BACKGROUND

Ambulatory medical devices (AMDs), such as wearable medical devices or implantable medical devices (IMDs), have been used for monitoring patient health condition or disease states. Some AMDs are capable of delivering therapies of one or more modalities to the patient. For example, implantable cardioverter-defibrillators (ICDs) may be used to monitor for certain abnormal heart rhythms and to deliver electrical energy to the heart to correct the abnormal rhythms. Some IMDs may be used to monitor chronic worsening of cardiac hemodynamic performance, such as due to congestive heart failure (CHF), and to provide cardiac stimulation therapies, including cardiac resynchronization therapy (CRT) to correct cardiac dyssynchrony within a ventricle or between ventricles.

Some AMDs can chronically record physiological data for monitoring cardiac arrhythmia. One type of cardiac arrhythmia is atrial fibrillation (AF), recognized as the most common clinical arrhythmia affecting millions of people. During AF, disorganized electrical pulses originated from regions in or near an atrium may lead to irregular conductions to ventricles, thereby causing inappropriately fast and irregular heart rate. AF may be paroxysmal that may last from minutes to days before it stops by itself. Persistent AF may last for over a week and typically requires medication or other treatment to revert to normal sinus rhythm. AF is permanent if a normal heart rhythm cannot be restored with treatment. AF may be associated with stroke and requires anticoagulation therapy.

Timely detection of atrial arrhythmia may be clinically important for assessing cardiac function. Atrial tachyarrhythmia may be characterized by fast atrial rate and irregular ventricular rates. However, irregular ventricular rates can be a caused by confounding factors such as respiration-mediated sinus arrhythmia, and affect atrial arrhythmia detection specificity. Inappropriate atrial arrhythmia detection may have adverse impact on patient outcome.

### OVERVIEW

This document discusses, among other things, systems, devices, and methods for detecting cardiac arrhythmia, such as atrial fibrillation (AF). A medical-device system includes an ambulatory monitor device and an arrhythmia analysis device communicatively coupled to each other. The ambulatory monitor device can sense a cardiac signal from the patient, and intermittently collect data segments (also referred to as data snippets) of the cardiac signal over a monitoring period in accordance with a data segment collection rate or schedule. The data segments each has a specific segment duration. The monitor device can transmit the intermittently collected data segments to the arrhythmia analysis device in accordance with a transmission schedule or in response to a trigger event. The arrhythmia analysis device can detect a cardiac arrhythmia from the intermittently collected data segments. The segment duration or the data segment collection rate or schedule may be adjusted based on a performance measure of the arrhythmia detection.

Example 1 is a system for monitoring a patient at risk of cardiac arrhythmia, the system comprising: an ambulatory monitor device configured to: sense a cardiac signal from the patient via a cardiac sensor; and intermittently collect data segments of the sensed cardiac signal in accordance with a data segment collection rate or schedule over a monitoring period, the data segments each having a segment duration; and an arrhythmia analysis device communicatively coupled to the ambulatory monitor device, the arrhythmia analysis device configured to detect a cardiac arrhythmia using intermittently collected data segments received from the ambulatory monitor device, wherein the ambulatory monitor device is configured to transmit the intermittently collected data segments to the arrhythmia analysis device in accordance with a transmission schedule or in response to a trigger event.

In Example 2, the subject matter of Example 1 optionally includes, the arrhythmia analysis device that can be configured to determine a confidence of the detection of the cardiac arrhythmia; and the ambulatory monitor device that can be configured to adjust the segment duration, including to increase the segment duration in response to the determined confidence falling below a confidence threshold, and to maintain or decrease the segment duration in response to the determined confidence exceeding a confidence threshold.

In Example 3, the subject matter of any one or more of Examples 1-2 optionally include the arrhythmia analysis device that can be configured to determine an instability of the detected cardiac arrhythmia; and the ambulatory monitor device that can be configured to adjust the segment duration, including to increase the segment duration in response to the determined instability exceeding an instability threshold, and to maintain or decrease the segment duration in response to the determined instability falling below the instability threshold.

In Example 4, the subject matter of any one or more of Examples 1-3 optionally include the ambulatory monitor device that can be configured to collect the data segments periodically with a data collection period longer than the segment duration.

In Example 5, the subject matter of any one or more of Examples 1-4 optionally include the ambulatory monitor device that can be configured to intermittently collect the data segments in accordance with a data collection schedule such that a time interval between at least two adjacent collections is longer than the segment duration.

In Example 6, the subject matter of any one or more of Examples 1-5 optionally include the ambulatory monitor device that can be configured to adjust the data segment collection rate or schedule, including to: intermittently collect the data segments in accordance with a first data segment collection rate or schedule over a first monitoring period; and intermittently collect the data segments in accordance with a second data segment collection rate or schedule different than the first data segment collection rate or schedule over a second monitoring period.

In Example 7, the subject matter of Example 6 optionally includes the arrhythmia analysis device that can be configured to determine a characteristic of the detected cardiac arrhythmia, wherein the ambulatory monitor device is configured to adjust the data segment collection rate or schedule based on the determined characteristic of the detected cardiac arrhythmia.

In Example 8, the subject matter of Example 7 optionally includes the characteristic of the detected cardiac arrhythmia that can include one or more of a heart rate or a duration of the detected cardiac arrhythmia.

In Example 9, the subject matter of any one or more of Examples 6-8 optionally include the arrhythmia analysis device that can be configured to determine a risk of the cardiac arrhythmia in the patient, and the ambulatory monitor device that can be configured to adjust the data segment collection rate or schedule based on the risk of the cardiac arrhythmia.

In Example 10, the subject matter of Example 9 optionally includes the arrhythmia analysis device that can be configured to determine the risk of the cardiac arrhythmia using a physiological signal different from the cardiac signal sensed from the patient.

In Example 11, the subject matter of any one or more of Examples 9-10 optionally includes the arrhythmia analysis device that can be configured to determine the risk of the cardiac arrhythmia based on a time of a day during the monitoring period.

In Example 12, the subject matter of any one or more of Examples 9-11 optionally include the arrhythmia analysis device that can be configured to determine the risk of the cardiac arrhythmia using an arrhythmia history of the patient.

In Example 13, the subject matter of any one or more of Examples 1-12 optionally include the arrhythmia analysis device that can be configured to detect the cardiac arrhythmia including an atrial fibrillation (AF) episode from each of the intermittently collected data segments.

In Example 14, the subject matter of any one or more of Examples 1-13 optionally include a user interface coupled to the arrhythmia analysis device, the arrhythmia analysis device configured to provide a notification about the detected cardiac arrhythmia to the user on the user interface.

In Example 15, the subject matter of Example 14 optionally includes the user interface that can be configured to receive a user adjudication of the detected cardiac arrhythmia as a true-positive or a false-positive detection, wherein the ambulatory monitor device is configured to adjust one or more of the segment duration or the data segment collection rate or schedule in response to the true-positive detection.

Example 16 is a method of monitoring a patient at risk of cardiac arrhythmia, the method comprising: sensing a cardiac signal from the patient using a cardiac sensor; intermittently collecting data segments of the sensed cardiac signal in accordance with a data segment collection rate or schedule over a monitoring period using an ambulatory monitor device, the data segments each having a segment duration; transmitting the intermittently collected data segments to an arrhythmia analysis device in accordance with a transmission schedule or in response to a trigger event; and detecting a cardiac arrhythmia using the intermittently collected data segments received from the ambulatory monitor device using the arrhythmia analysis device.

In Example 17, the subject matter of Example 16 optionally includes: determining a confidence of the detection of the cardiac arrhythmia; and adjusting the segment duration, including increasing the segment duration in response to the determined confidence falling below a confidence threshold, and maintaining or decreasing the segment duration in response to the determined confidence exceeding a confidence threshold.

In Example 18, the subject matter of any one or more of Examples 16-17 optionally include: determining an instability of the detected cardiac arrhythmia; and adjusting the segment duration, including increasing the segment duration in response to the determined instability exceeding an instability threshold, and maintaining or decreasing the segment duration in response to the determined instability falling below the instability threshold.

In Example 19, the subject matter of any one or more of Examples 16-18 optionally include, wherein intermittently collecting the data segments includes periodically collecting the data segments with a data collection period longer than the segment duration.

In Example 20, the subject matter of any one or more of Examples 16-19 optionally include, wherein intermittently collecting the data segments includes varying the data segment collection rate or schedule, such that the data segments are intermittently collected in accordance with a first data segment collection rate or schedule during a first monitoring period, and with a second data segment collection rate or schedule different than the first data segment collection rate or schedule during a second monitoring period different than the first monitoring period.

In Example 21, the subject matter of Example 20 optionally includes determining a characteristic of the detected cardiac arrhythmia including one or more of a heart rate or a duration of the detected cardiac arrhythmia, wherein varying the data segment collection rate or schedule is in accordance with the determined characteristic of the detected cardiac arrhythmia.

In Example 22, the subject matter of any one or more of Examples 20-21 optionally include receiving information about a risk of the cardiac arrhythmia in the patient, wherein varying the data segment collection rate or schedule is in accordance with the risk of the cardiac arrhythmia.

In Example 23, the subject matter of any one or more of Examples 16-22 optionally include: receiving a user adjudication including a true-positive or a false-positive designation of the detected cardiac arrhythmia; and adjusting one or more of the segment duration or the data segment collection rate or schedule in response to a true-positive designation of the detected cardiac arrhythmia.

This Overview is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIGS. 2A-2C illustrate generally examples of intermittent data snippet collection in accordance with respective data collection rates or schedules.

FIG. 3 illustrates generally an example of an arrhythmia detection system configured to monitor a patient at risk of cardiac arrhythmia.

FIG. 4 is a flow chart illustrating an example of a method of assessing a patient's risk of cardiac arrhythmia such as using the system as illustrated in FIG. 3.

FIG. 5 illustrates generally a block diagram of an example machine upon which any one or more of the techniques (e.g., methodologies) discussed herein may perform.

### DETAILED DESCRIPTION

Some AMDs, such as implantable cardiac monitors (ICMs), are capable of chronically recording cardiac information, optionally along with other physiological data, from a patient. The recorded data can be stored in an ICM, and transmitted to an external computing device, such as a remote server, where a clinician can remotely monitor patient health status. The external computing device can additionally or alternatively analyze the data recorded by and transmitted from the ICM, and detect or predict a cardiac event, such as cardiac arrhythmia or progression of heart failure. The external computing device generally has resources and computing power sufficient to analyze a large volume of data. In some cases where real-time event detection is not required (e.g., non-lethal arrhythmias such as chronic AF, or worsening of chronic heart failure), the external computing device can perform retrospective batch-mode analysis of data transmitted from the ICM.

An ICM can include data acquisition circuitry to collect cardiac information including, for example, electrocardiograms or ECGs, subcutaneous electrograms or EGMs, or other physiological information. The cardiac or physiological information may be collected constantly or periodically. The collected data can be stored in an onboard memory before a scheduled transmission to the external computing device (e.g., a remote server). In an example of monitoring chronic AF, an ICM can collect a large number of data segments (also referred to as data snippets) over a long time period, where each data segment has a duration sufficiently long (e.g., 4-5 minutes) to allow the external computing device to reliably detect therefrom a presence or absence of an AF event. However, as ICMs generally have limited battery longevity, memory capacity, and communication bandwidth, onboard collection, storage, and transmission of a large volume of data can be technically challenging. On the other hand, with advanced computing technology and computationally intensive and high-performance arrhythmia detection algorithms, the external computing device can reliably detect arrhythmia using a much shorter data snippet without substantially compromising detection sensitivity or specificity. For example, instead of using a 4-minute long data snippet, the external computing device can use machine learning-based algorithms to detect AF events from a snippet of one-minute long or even shorter. From the battery longevity and memory usage standpoint, the number of snippets that an ICM can collect and the size (i.e., duration) of a snippet are engineering tradeoffs. For example, sampling and storing one snippet of a 4-minute long ECG would consume approximately the same battery power and takes approximately the same amount of memory space as sampling and storing four 1-minute long ECG snippets, or eight 30-second long ECG snippets, or sixteen 15-second long ECG snippets, or thirty two 7.5-second long ECG snippets, etc. If the external computing device can detect AF using a shorter ECG snippet with adequate performance (e.g., sensitivity and specificity above acceptable thresholds, or false positive rate or false negative rate below acceptable thresholds), then more snippets can be collected and transmitted to the external computing device without additional battery power or onboard resources requirement. Because more snippets are generally acquired over a longer monitoring period, there is an increased likelihood of capturing more AF events particularly in patients with chronic or persistent AF. For example, if the external computing device is capable of detecting AF with sufficient accuracy from a 30-second ECG snippet, eight of such snippets can be provided to the external computing device, get analyzed by the detection algorithms and reviewed by the clinician, instead of only one 4-minute snippet which would consume approximately the same amount of batter power and resources of the ICM. With proper data snippet collection schedules, the eight snippets can cover a monitoring period greater than four minutes, and more AF events are likely to be detected.

The present inventors have recognized an unmet need to optimize data snippet collection and transmission schedules based on the capability of the external computing device so that a longer monitoring period and potentially more arrhythmic events can be detected without substantially compromising the battery longevity or exhausting the onboard resources of ICM. Disclosed herein are systems, devices, and methods for detecting cardiac arrhythmia, such as atrial fibrillation (AF). A medical-device system includes an ambulatory monitor device and an arrhythmia analysis device communicatively coupled to each other. The ambulatory monitor device can sense a cardiac signal from the patient, and intermittently collect data segments of the sensed cardiac signal in accordance with a data segment collection rate or schedule over a monitoring period. The data segments each has a specific segment duration. The monitor device can transmit the intermittently collected data segments to the arrhythmia analysis device in accordance with a transmission schedule or in response to a trigger event. The arrhythmia analysis device can detect a cardiac arrhythmia from the intermittently collected data segments. The segment duration or the data segment collection rate or schedule may be adjusted based on a performance measure of the arrhythmia detection.

The systems, devices, and methods discussed in this document may improve the medical technology of device-based arrhythmia detection, particularly detection of AF events. In accordance with various embodiments, intermittent data snippets collection, storage, and transmission and optimized snippet size (i.e., data segment duration) at the ICM can take the best advantage of computationally intensive and high-performance arrhythmia detection capabilities of the external computing device, facilitate clinician review of patient data over an extended monitoring period, and allow for more AF events potentially to be detected, yet without overusing the onboard resources and battery power of the ICMs. Consequently, medical resources may be better aligned to serve more patients, and the patient management cost in a healthcare facility may be reduced.

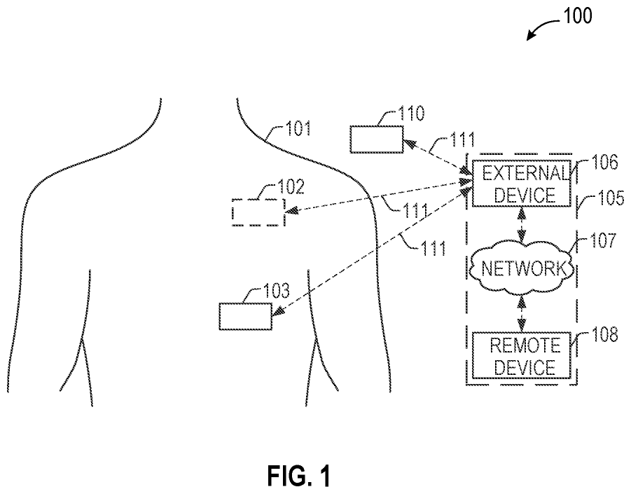

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more ambulatory medical devices, an external system 105, and a communication link 111 providing for communication between the one or more ambulatory medical devices and the external system 105. The one or more ambulatory medical devices can include an implantable medical device (IMD) 102, a wearable medical device (WMD) 103, or one or more other implantable, leadless, subcutaneous, external, wearable, or ambulatory medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 can include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

The 1 MB 102 can include an assessment circuit configured to detect or determine specific physiologic information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the 1 MB 102 or one or more of the other ambulatory medical devices, etc. In some examples, therapy can include cardiac resynchronization therapy for rectifying dyssynchrony and improving cardiac function in heart failure patients. In other examples, the 1 MB 102 can include a drug delivery system, such as a drug infusion pump to deliver drugs to the patient for managing arrhythmias or complications from arrhythmias, hypertension, or one or more other physiologic conditions. In other examples, the IMD 102 can include one or more electrodes configured to stimulate the nervous system of the patient or to provide stimulation to the muscles of the patient airway, etc.

The WMD 103 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 105 can include a dedicated hardware/software system, such as a programmer, a remote server-based patient management system, or alternatively a system defined predominantly by software running on a standard personal computer. The external system 105 can manage the patient 101 through the 1 MB 102 or one or more other ambulatory medical devices connected to the external system 105 via a communication link 111. In other examples, the IMD 102 can be connected to the WMD 103, or the WMD 103 can be connected to the external system 105, via the communication link 111. This can include, for example, programming the 1 MB 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information can include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected physiological event can be prioritized using a similarity metric between the physiological data associated with the detected physiological event to physiological data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiological events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

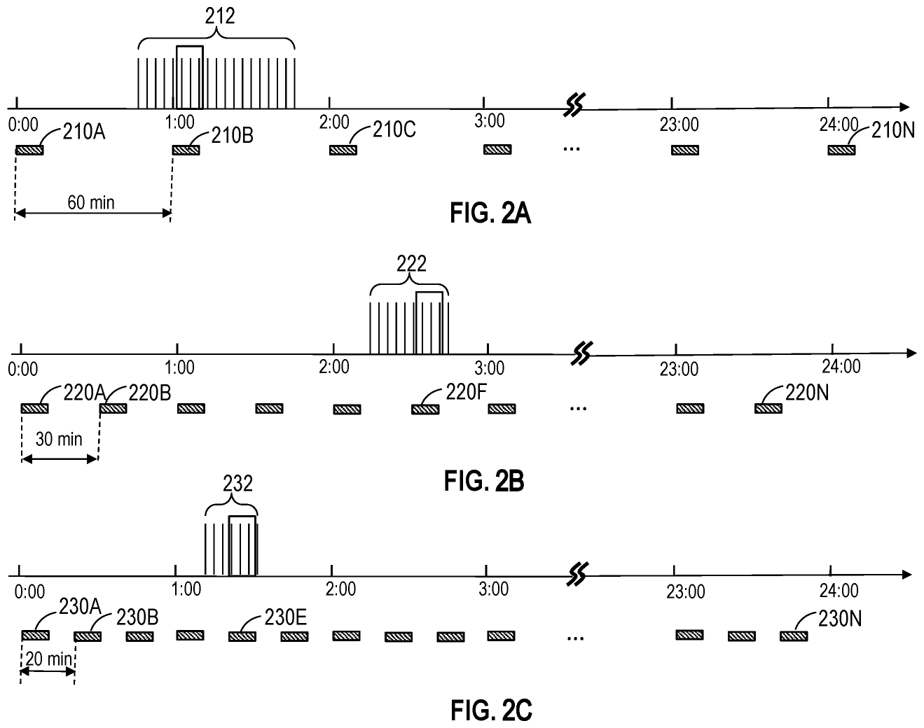

FIGS. 2A-2C illustrate generally examples of intermittent data snippet collection in accordance with respective data collection schedules based on characteristics of underlying cardiac rhythms, such as durations of AF episodes. The data snippets can be collected intermittently (as opposed to continuously) over a specified monitoring period, such as a 24-hour period in a non-limiting example as shown in FIGS. 2A-2C. In certain examples, the monitoring period can be a pre-determined period that the patient is more prone to arrhythmia (e.g., AF), which can be determined based on patient history, time of a day, or indications from certain physiological sensors (e.g., activity sensors). The data snippet collection schedule may include a snippet size (i.e., the duration of a data segment) and a snippet collection rate (e.g., the number of snippets per unit time for periodic snippet collection) or scheduled timings to initiate collection of data snippets (which can be non-periodic snippet collection). FIG. 2A illustrates an example of periodic collection of data snippets 210A, 210B, . . . , 210N over a 24-hour monitoring period at a rate of one snippet every 60 minutes. Each snippet is 10-second long. To ensure intermittent rather than continuous data collection, the snippet size (e.g., 10 seconds in the present example) is set to be shorter than the time interval between at least two adjacent data snippets. For periodic snippet collection, the snippet size is set to be shorter than the snippet collection period (e.g., 60 minutes in the present example). The periodic collection results in 24 snippets in the 24-hour monitoring period. Such data snippet collection schedule would guarantee to capture at least one 10-second “snapshot” of an AF episode that lasts 60 minutes or longer. For example, the data snippet 210B that begins at 1:00 can capture a 10-second portion of the 60-minute long AF episode 212.

FIG. 2B illustrates another example of periodic collection of data snippets 220A, 220B, . . . , 220N over the same 24-hour monitoring period as shown in FIG. 2A. Each snippet is also 10-second long, but the snippets are collected at a rate of one snippet every 30 minutes, resulting in a total of 48 snippets in the 24-hour monitoring period. The data snippet collection schedule would guarantee to capture at least one 10-second “snapshot” of an AF episode that lasts 30 minutes or longer. For example, the data snippet 220F that begins at 2:30 can capture a 10-second portion of the 30-minute long AF episode 222. Likewise, FIG. 2C illustrates yet another example of periodic collection of data snippets 230A, 230B, . . . , 230N over the same 24-hour monitoring period as shown in FIGS. 2A and 2B. The 10-second snippets are collected at a rate of one snippet every 20 minutes, resulting in a total of 72 snippets in the 24-hour monitoring period. The data snippet collection schedule would guarantee to capture at least one 10-second “snapshot” of an AF episode that lasts 20 minutes or longer. For example, the data snippet 230E that begins at 1:20 can capture a 10-second portion of the 20-minute long AF episode 232. Similarly, in an example where 10-second snippets are collected at a rate of one snippet every four minutes (such as to capture a “snapshot” of an AF episode lasting at least four minute), a total of 360 snippets can be collected over a 24-hour monitoring period; or in another example where 10-second snippets are collected at a rate of one snippet every two minutes (such as to capture a “snapshot” of an AF episode lasting at least two minute), a total of 720 snippets can be collected over a 24-hour monitoring period.

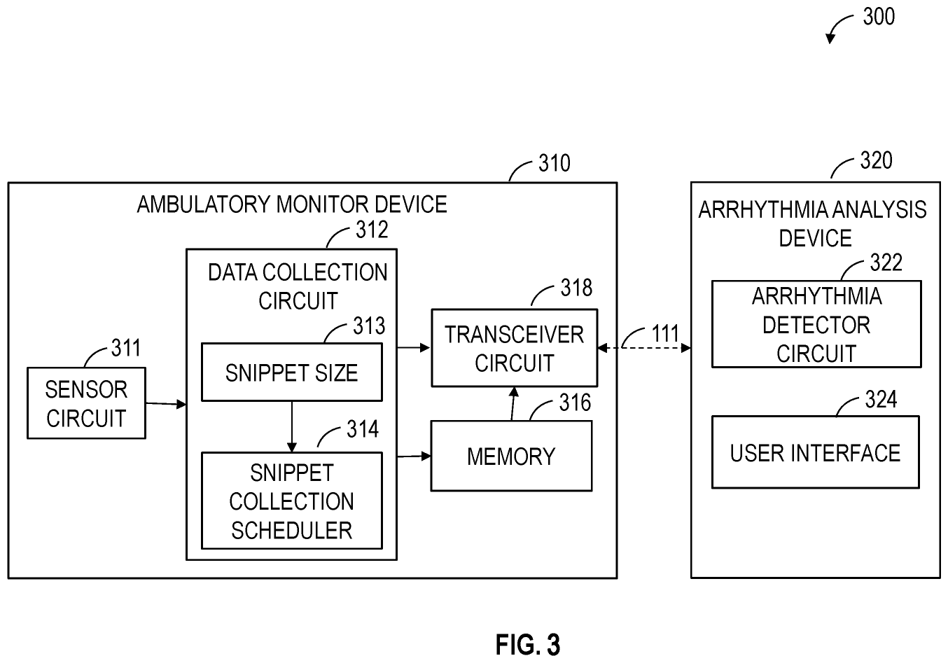

As described above, a data snippet collection schedule includes a snippet size and a snippet collection rate (for periodic snippet collection) or timings to initiate data snippet collection. In an example, the snippet size (e.g., 10 seconds as shown in the examples above) can be determined based on the capability of an external computing device (such as one or more of the devices in the external system 105) to detect a presence or absence of AF events with sufficient accuracy from a data snippet. In an example, the snippet collection rate or timing schedules can be determined based on characteristics (such as heart rate or durations) of the AF episodes. For periodic snippet collection, as shown in FIGS. 2A-2C, setting the snippet collection period to be equal to or shorter than the AF episode duration would guarantee to capture at least a portion of said AF episode. In various examples, the snippet collection rate or timing schedules can be determined based on an estimate of the AF episode duration, arrhythmia risk or arrhythmia history of the patient, a time of a day, or other physiological information. Examples of determining the data snippet collection schedules are discussed below with reference to FIG. 3.

The intermittently collected data snippets, such as 210A-210N, 220A-220N, or 230A-230N, can be stored in a memory of the IMD 102 or the WMD 103. The stored data snippets can be transmitted to the external computing device such as one or more of the devices in the external system 105 for further analysis (e.g., to detect the presence of absence of AF events), or to be presented to a clinician. The stored data snippets can be transmitted via a communication link in accordance with a transmission schedule or in response to a trigger event. As previously discussed, optimizing data snippet size and intermittent snippet collection schedules as described herein in accordance to various embodiments can take better advantage of the computationally intensive and high-performance arrhythmia detection capabilities of the external computing device, allow a clinician to review data over an extended monitoring period, and potentially detect more AF events during the extended monitoring period, yet without overusing the onboard resources and battery power of the ICM.

Although the discussion of intermittent data snippet collection in the present document is focused on snippet of cardiac signals for AF monitoring, the systems, devices, and techniques described herein can be applied to intermittent data snippet collection of other physiological information and used for detecting other physiological events or conditions.

FIG. 3 illustrates generally an example of an arrhythmia detection system 300 configured to monitor a patient at risk of cardiac arrhythmia, such as atrial fibrillation (AF). Portions of the system 300 may be included in the patient management system 100. The system 300 may include an ambulatory monitor device 310 and an arrhythmia analysis device 320. The ambulatory monitor device 310 can be an example of the IMD 102 or the WMD 103. In an example, the ambulatory monitor device 310 can include a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein. In some examples, the ambulatory monitor device 310 may include circuit sets comprising one or more other circuits or sub-circuits. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

As illustrated in FIG. 3, the ambulatory monitor device 310 can include a sensor circuit 311, a data collection circuit 312, a memory 316, and a transceiver circuit 318. The sensor circuit 311 may include circuitry configured to sense a physiologic signal from a patient such as via one or more implantable, wearable, or otherwise ambulatory sensors or electrodes associated with the patient. The sensors may be incorporated into, or otherwise associated with an ambulatory medical device, such as the IMD 102 or the WMD 103. In some examples, the sensors may be incorporated into an implantable cardiac monitor (ICM) device configured for subcutaneous implantation. Examples of the physiologic signals may include cardiac signals including, for example, surface electrocardiography (ECG), subcutaneous ECG, or intracardiac electrogram (EGM), thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal such as sensed by an ambulatory accelerometer or acoustic sensors, physiologic response to activity, apnea hypopnea index, one or more respiration signals such as a respiration rate signal or a tidal volume signal, brain natriuretic peptide (BNP), blood panel, sodium and potassium levels, glucose level and other biomarkers and bio-chemical markers, among others. The sensor circuit 310 may include one or more sub-circuits to digitize, filter, or perform other signal conditioning operations on the received physiologic signal.

The data collection circuit 312 can collect data snippets of a physiologic signal received from the sensor circuit 311. As described above with reference to FIG. 2, the data snippets are short segments of the sensed signal with specific data length, also referred to as a snippet size 313. The snippet size 313 can be a programmable value that can be set by a user via a programmer device. The snippet size 313 can be within a specific range, such as approximately between 10 and 60 seconds. By way of example and not imitation, the snippet size can be set to 10 seconds, as shown in FIGS. 2A-2C.

In some examples, the data collection circuit 312 can determine the snippet size 313 based on the capability of the arrhythmia analysis device 320 to detect arrhythmic events (e.g., AF events) with sufficient accuracy from a data snippet. A detection performance of the arrhythmia analysis device 320 can be assessed to determine if a particular data snippet size is sufficient. Examples of the detection performance may include sensitivity, specificity, false positive rate, or false negative rate. If the detection performance of the arrhythmia analysis device 320 satisfies a criterion (e.g., the sensitivity and/or the specificity exceed respective thresholds, or the false positive rate and/or false negative rate are below respective thresholds), then the snippet size 313 can be reduced; otherwise, the snippet size 313 can be maintained at its present value, or be increased to improve the arrhythmia detection performance of the arrhythmia analysis device 320.

In some examples, the data collection circuit 312 can adjust the snippet size 313 based on a confidence of the arrhythmia detection made by the arrhythmia analysis device 320. Generally, a larger snippet size would result in a higher detection confidence, and vice versa. On the other hand, a shorter snippet size is generally preferred over a longer one at least because a longer snippet size may correspond to a smaller number of snippets being collected during the monitoring period due to the engineering tradeoff for a given battery power, as described above. As such, an “optimal” snippet size can be one that is short but does not significantly comprise the arrhythmia detection confidence. In an example where the arrhythmia detection is based on a comparison of a signal metric value to a threshold, the detection confidence can be based on a deviation of the signal metric from the threshold. If the detection confidence falls below a confidence threshold, then the snippet size 313 can be increased (e.g., from 10 seconds to 20 seconds). If the detection confidence exceeds the confidence threshold, then snipe size 313 can be maintained at its present value or be decreased.

In additional or alternative to the cardiac arrhythmia detection performance or confidence as described above, in some examples, the snippet size 313 can be adjusted based on an instability of the cardiac arrhythmia detected by the arrhythmia analysis device 320. Such instability indicates a degree of change or a rate of change in certain properties of the detected cardiac arrhythmia. If the arrhythmia instability exceeds an instability threshold, then the snippet size 313 can be increased (e.g., from 10 seconds to 20 seconds); if the arrhythmia instability falls below the instability threshold, then the present snipe size 313 can be maintained or decreased. For example, a substantial change (e.g., exceeding a threshold value) in the heart rate variability during AF may indicate an instable AF or a new type of AF episodes, then the snippet size can be extended so that more data is collected and ultimately made available to the user (for reviewing the data snippet) or to the arrhythmia analysis device to further analyze the data snippet.

The data collection circuit 312 can collect data snippets intermittently over a monitoring period, such as a 24-hour period as illustrated in FIGS. 2A-2C. The data collection circuit 312 can include a snippet collection scheduler 314 that determines a schedule to collect data snippets from the sensed physiologic signal. The snippet collection schedule can be represented by timings to initiate collection of data snippets. In an example of periodic snippet collection, the snippet collection schedule can be represented by a snippet collection rate (e.g., N snippets per unit time) or equivalently a snippet collection period (e.g., one snippet every X minutes). The snippet collection rate or snippet collection period can be a programmable value that can be set by a user via a programmer device. FIGS. 2A-2C show the examples where the snippet collection period X is set to 60, 30, or 20 minutes. By way of example and not limitation, the snippet collection period X can be within a range of approximately between 2 minutes to 60 minutes. To ensure intermittent rather than continuous data collection, the time interval between at least two adjacent data snippets, or the data collection period in the case of periodic snippet collection, can be set to be longer than the snippet size 313.

In various examples, the snippet collection scheduler 314 can establish a variable data snippet collection rate or a variable timing schedule for collecting data snippets during the monitoring period, such that during a first monitoring period, data snippets are intermittently collected at a first collection rate or schedule, and during a second monitoring period subsequent to the first monitoring period, data snippets are intermittently collected at a second collection rate or schedule different than the first collection rate or schedule. In an example, the snippet collection scheduler 314 can adjust the data snippet collection rate or timing schedule based on a characteristic of the cardiac arrhythmia detected by the arrhythmia analysis device 320. Examples of such characteristics can include heart rate, or arrhythmia duration, among others. In an example, the snippet collection scheduler 314 can adjust the data snippet collection rate or timing schedule based on an adjudication of an arrhythmia event (e.g., an AF event) detected by the arrhythmia analysis device 320. For example, a user (e.g., a clinician) can review the snippet data and adjudicate the AF detection as either a true positive (TP) or a false positive (FP) detection, and the snippet collection scheduler 314 can increase the snippet collection rate (or equivalently decrease the snippet collection period) when a TP detection is adjudicated. In another example, the snippet collection scheduler 314 can adjust the data snippet collection rate or timing schedule based on patient risk of having a cardiac arrhythmia. The arrhythmia risk can be determined using arrhythmia history of the patient, a time of a day, or physiological information such as sensed from a physiologic sensor. The physiologic sensor used for determining the arrhythmia risk can be different from the sensors used for producing the cardiac signals from which data snippets are generated. The arrhythmia risk can be determined by the ambulatory monitor device 310. Alternatively, the arrhythmia risk can be determined by the arrhythmia analysis device 320, and provided to the ambulatory monitor device 310 for use in adjusting the collection rate or timing schedule.

Data snippets collected by the data collection circuit 312 can be stored in the memory 316. The ambulatory monitor device 310 can include a transceiver circuit 318 configured to transmit at least a portion of the stored data snippets to the arrhythmia analysis device 320 via a communication link 111. The arrhythmia analysis device 320 can be an example of the external device 106 or the remote device 108 of the external system 105. In some examples, the transceiver circuit 318 can transmit the data snippets in accordance with a transmission schedule (e.g., automatic periodic transmission) or in response to a trigger event (e.g., a commanded transmission in response to a user command). By way of example and not limitation, the transmission schedule can include one daily transmission of all or at least a portion of the data snippets collected over a 24-hour period. For example, the transceiver circuit 318 can transmit, on a daily basis (i.e., one transmission session per day), up to 24 snippets collected according to the schedule as illustrated in FIG. 2A, up to 48 snippets collected according to the schedule as illustrated in FIG. 2B, or up to 72 snippets collected according to the schedule as illustrated in FIG. 2C, or other numbers of snippets in accordance with respective snippet collection rates or schedules (e.g., 360 snippets corresponding to a rate of one snippet every four minutes, or 720 snippets corresponding to a rate of one snippet every two minutes). In some examples, the transceiver circuit 318 can adjust the transmission schedule for transmitting the data snippets to the arrhythmia analysis device 320. For example, depending on the amount of daily collected snippets, the transceiver circuit 318 can increase the transmission schedule from one transmission session per day to two transmission sessions per day, or decrease the transmission schedule from one transmission session per day to one transmission session every other day. In some examples, the adjustment of transmission schedule can include a change of time of a day for data transmission.

The arrhythmia analysis device 320 includes an arrhythmia detector circuit 322 and a user interface 324. The arrhythmia detector circuit 320 cam detect a cardiac arrhythmia (e.g., AF) from the intermittently collected data segments received from the ambulatory monitor device 310 using reconfigurable, computationally intensive and high-performance arrhythmia detection algorithms implemented therein. In an example, the arrhythmia detector circuit 320 can detect a presence or absence of AF from each of the intermittently collected data snippet. In an example, the AF event may be detected using ventricular rate stability. The arrhythmia detector circuit 320 may detect an AF event if the ventricular rate variability exceeds a threshold or is within a value range. In some examples, the AF detector 234 may detect an AF event using morphology of cardiac signals. In another example, the arrhythmia detector circuit 320 may detect an AF event using a ventricular rate pattern of consecutive decrease in ventricular rate. The ventricular rate pattern includes a pair of consecutive ventricular rate changes. Both ventricular rate changes are negative, referred to as a “double decrement” ventricular rate pattern. A double-decrement ratio, which represents a prevalence of the double decrement ventricular rate pattern over a specified time period or over a plurality of ventricular beats, may be computed, and used to detect AF. Krueger et al. U.S. patent application Ser. No. 14/825,669, entitled “ATRIAL FIBRILLATION DETECTION USING VENTRICULAR RATE VARIABILITY,” refers to double decrement pattern in ventricular heart rate and its use in atrial arrhythmia detection, the disclosure of which is incorporated by reference herein in its entirety. In yet another example, the arrhythmia detector circuit 320 may detect an AF event using a ventricular rate cluster, represented by a statistical distribution or a histogram of ventricular rate or cycle length over multiple cardiac cycles. The ventricular rate cluster indicates regularity of ventricular rates of cardiac cycle lengths. Patients with AF are typically presented with irregular ventricular contractions. However, premature atrial contractions (PACs) may occur at irregular intervals. When PACs conduct to the ventricle, they may produce irregular ventricular rates, resulting in different ventricular clusters than AF. As such, the ventricular rate clusters may be used to distinguish frequent PACs from an AF event. Perschbacher et al. U.S. patent application Ser. No. 15/864,953 entitled “ATRIAL FIBRILLATION DISCRIMINATION USING HEART RATE CLUSTERING,” refers to histogram clusters of ventricular rates and their use in discriminating between AF and non-AF events, the disclosure of which is incorporated by reference herein in its entirety. In yet another example, the arrhythmia detector circuit 320 may detect an AF event using a metric representing the occurrence of various beat patterns of the cycle lengths or heart rates. In an example, the statistical measure includes an atrioventricular (AV) conduction block metric indicating a presence or degree of conduction abnormality during a sinus rhythm, such as a Wenckebach score representing the prevalence of Wenckebach block over a time period. Examples of the Wenckebach detector may be based on a repetitiveness indictor of various beat patterns of the cycle lengths or heart rates, such as discussed in Perschbacher et al. U.S. patent application Ser. No. 15/786,824 entitled “SYSTEMS AND METHODS FOR ARRHYTHMIA DETECTION,” the disclosure of which is incorporated by reference herein in its entirety.

In some examples, the arrhythmia detector circuit 320 can determine an confidence of the arrhythmia detection, such as an AF confidence. The confidence can have a categorical or numerical value. In an example, an AF confidence score can be determined as a function of an atrial peak intensity within an atrial detection window. In an example, an AF confidence score is inversely proportional to the atrial peak intensity, such that a higher AF confidence score may be assigned to an AF event that has a lower atrial peak intensity in the ensemble-averaged cardiac electrical signal. In another example, an AF confidence score can be determined as a function of the signal power of the portion of the ensemble-averaged cardiac electrical signal within the atrial detection window.

As described above, the performance of AF detection (e.g., sensitivity and specificity, or false positive rate and false negative rate) and/or the AF confidence indication can be provided as feedback to the ambulatory monitor device 10, via the communication link 111, to adjust snippet size and/or the data snippet collection rate or the timing schedule for collecting data snippets.

The user interface 324 may include an input device and an output device. The input device may receive a user's programming input for the ambulatory monitor device 310, such as snippet size and parameters related to snippet collection schedules, and/or user programming input for the arrhythmia detection at the arrhythmia analysis device 320. The input device may include a keyboard, on-screen keyboard, mouse, trackball, touchpad, touch-screen, or other pointing or navigating devices. The input device may enable a system user to program the parameters used for sensing the physiologic signals, detecting the arrhythmias, and generating alerts, among others. The output device may generate a human-perceptible presentation of the detected cardiac arrhythmic events, such as the AF events. The output device may include a display for displaying the sensed physiologic information, intermediate measurements or computations, the detected AF events, or the AF confidence indicators for the AF events, among others. The output unit may include a printer for printing hard copies of the detection information. The information may be presented in a table, a chart, a diagram, or any other types of textual, tabular, or graphical presentation formats. The presentation of the output information may include audio or other media format to alert the system user of the detected arrhythmic events. In an example, the output device may generate alerts, alarms, emergency calls, or other forms of warnings to signal the system user about the detected arrhythmic events.

In some examples, the system 300 may include a therapy unit that can deliver an antiarrhythmic therapy to the patient in response to the detection of the cardiac arrhythmia. The therapy unit can be included in the ambulatory monitor device 310; or alternatively in a separate therapeutic system. Examples of the therapy may include electrostimulation therapy delivered to the heart, a nerve tissue, other target tissues, a cardioversion therapy, a defibrillation therapy, or drug therapy including delivering drug to a tissue or organ. In some examples, an existing therapy or treatment plan may be modified to treat the detected arrhythmia, such as modify patient follow-up schedule, or adjust a stimulation parameter or drug dosage.

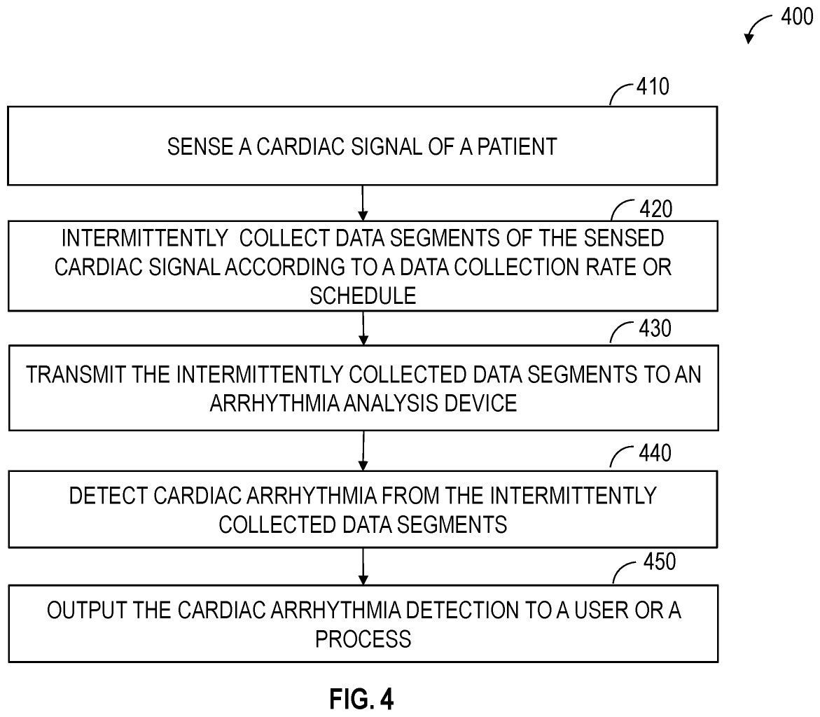

FIG. 4 is a flow chart illustrating an example of a method 400 for monitoring a patient at risk of cardiac arrhythmia, such as atrial fibrillation (AF), using data segments (or data snippets) intermittently collected from the patient. The method 400 may be implemented and executed in an ambulatory medical device such as the IMD 102 or the WMD 103, or in the external system 105. In an example, the method 400 may be implemented in and executed by the arrhythmia detection system 300.

The method 400 begins at 410, where a physiologic signal may be sensed from the patient using one or more implantable, wearable, or otherwise ambulatory sensors or electrodes included in or associated with the 1 MB 102, the WMD 103, or the ambulatory monitor device 310. Alternatively, the physiologic signal may be retrieved from a storage device (e.g., an electronic medical record system) that stores physiologic signals recorded from a patient. In an example, the physiologic signal may include a cardiac electrical signal, such as an ECG or an intracardiac EGM. In some examples, the physiologic signal may include thoracic or cardiac impedance signal, arterial pressure signal, pulmonary artery pressure signal, left atrial pressure signal, RV pressure signal, LV coronary pressure signal, heart sounds or endocardial acceleration signal, physiologic response to activity, apnea hypopnea index, one or more respiration signals such as a respiration rate signal or a tidal volume signal, among others. The sensed physiologic signal may be pre-processed, including one or more of signal amplification, digitization, filtering, or other signal conditioning operations.

At 420, data segments of the sensed cardiac signal may be intermittently collected in accordance with a data snippet collection rate or schedule over a monitoring period, such as using the data collection circuit 312. The intermittently collected data can be in the form of data segments or snippets with specific data length, also known as snippet size. The snippet size can be a programmable value that can be set by a user via a programmer device. As describe above with reference to FIG. 3, the snippet size can be determined based on how an arrhythmia analysis device, such as the arrhythmia analysis device 320 or an offline arrhythmia analyzer in the external system 105, can accurately detect a presence or absence of a target arrhythmic event (e.g., AF events). Additionally or alternatively, the snippet size can be determined based on a confidence of the cardiac arrhythmia detection. In some examples, the snippet size can be determined based on an instability of the cardiac arrhythmia representing a degree of change or a rate of change in certain properties of the detected cardiac arrhythmia.

The intermittent collection of data snippets can include, in an example, periodic snippet collection at a specific data (snippet) collection rate, represented by the number of snippets to be collected per unit time. The periodic snippet collection can alternatively be represented by a snippet collection period (i.e., one snippet every X minutes). FIGS. 2A-2C show the examples where the snippet collection period is set to 60, 30, or 20 minutes. In some examples, the intermittent collection can include non-periodic collection schedules. To ensure intermittent rather than continuous data collection, the time interval between at least two adjacent data snippets, or the data collection period in the case of periodic snippet collection, can be set to be longer than the snippet size.

In various examples, the intermittent data snippet collection can be carried out at a variable data snippet collection rate or a variable timing schedule for collecting data snippets during the monitoring period, such that the data segments are collected in accordance with a first data snippet collection rate or schedule during a first monitoring period, and with a second data snippet collection rate or schedule different than the first data snippet collection rate or schedule during a second monitoring period different than the first monitoring period. The adjustment of the snippet collection rate or timing schedule for collecting data snippets can be based on a characteristic of the cardiac arrhythmia, such as heart rate, arrhythmia duration, among others. In an example, the data snippet collection rate or timing schedule can be adjusted based on arrhythmia adjudication of an arrhythmia episode, such that, for example, the snippet collection rate can be increased in response to true-positive (TP) adjudication of the arrhythmia. In another example, the data snippet collection rate or timing schedule can be adjusted based on patient risk of having a cardiac arrhythmia, which can be determined using information of one or more of arrhythmia history of the patient, a time of a day, or physiological information such as sensed from a physiologic sensor.

At 430, the intermittently collected data segments can be transmitted to an arrhythmia analysis device for further analysis and/or for expert review or adjudication. The transmission of data segments can follow a transmission schedule or in response to a trigger event.

At 440, the intermittently collected data segments can be analyzed by the arrhythmia analysis device to detect a cardiac arrhythmia of a particular type, such as AF. Reconfigurable, computationally intensive and high-performance arrhythmia detection algorithms can be used to detect a presence or absence of the arrhythmia (e.g., AF) from each of the intermittently collected data snippet. In some examples, a confidence indicator may be determined for the detected arrhythmia episode.

Based on the arrhythmia detection performance at 440, one or more of the snippet size or the data snippet collection rate or timing schedule can be adjusted. For example, performance of AF detection at 440, including sensitivity and specificity, or false positive rate and false negative rate, may be computed. Such performance measures, and/or the confidence indictors associated with the detected arrhythmia episodes, may be used as feedback to adjust snippet size and/or the data snippet collection rate or the timing schedule for collecting data snippets.

At 450, information about the detected cardiac arrhythmia may be presented to a user or to a process. Alert notification about the detected cardiac arrhythmia may be present to the user on a user interface. In an example, arrhythmia adjudication may be received from the user. In some examples, a therapy may be delivered to the patient in response to the detection of the cardiac arrhythmia, or an existing therapy or treatment plan may be modified to treat the detected arrhythmia.

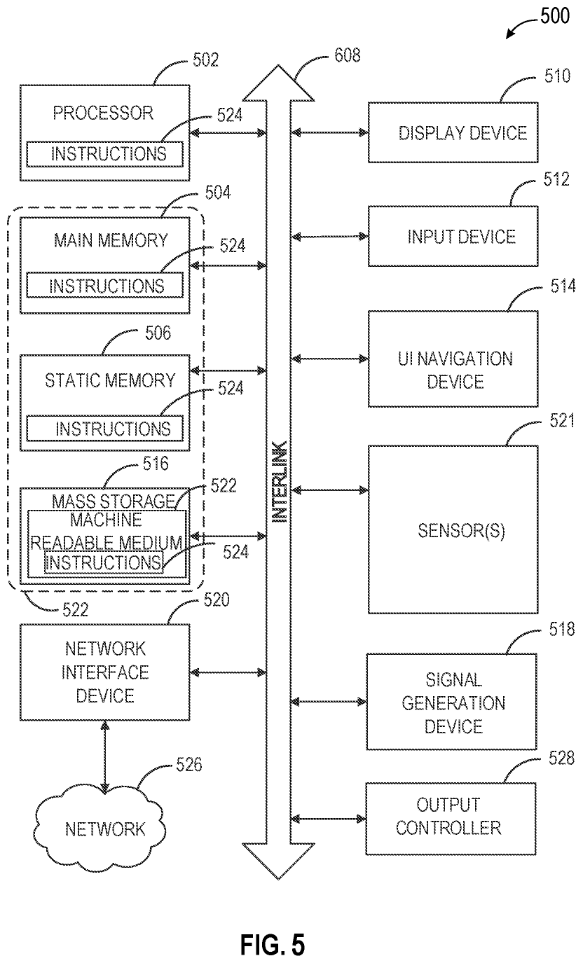

FIG. 5 illustrates generally a block diagram of an example machine 500 upon which any one or more of the techniques (e.g., methodologies) discussed herein may perform. Portions of this description may apply to the computing framework of various portions of the patient management system 100 or the arrhythmia detection system 300.

In alternative embodiments, the machine 500 may operate as a standalone device or may be connected (e.g., networked) to other machines. In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 500 may include a hardware processor 502 (e.g., a central processing unit (CPU), a graphics processing unit (GPU), a hardware processor core, or any combination thereof), a main memory 504 and a static memory 506, some or all of which may communicate with each other via an interlink (e.g., bus) 508. The machine 500 may further include a display unit 510 (e.g., a raster display, vector display, holographic display, etc.), an alphanumeric input device 512 (e.g., a keyboard), and a user interface (UI) navigation device 514 (e.g., a mouse). In an example, the display unit 510, input device 512 and UI navigation device 514 may be a touch screen display. The machine 500 may additionally include a storage device (e.g., drive unit) 516, a signal generation device 518 (e.g., a speaker), a network interface device 520, and one or more sensors 521, such as a global positioning system (GPS) sensor, compass, accelerometer, or other sensor. The machine 500 may include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 516 may include a machine readable medium 522 on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the storage device 516 may constitute machine-readable media.

While the machine-readable medium 522 is illustrated as a single medium, the term “machine readable medium” may include a single medium or multiple media (e.g., a centralized or distributed database, and/or associated caches and servers) configured to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for monitoring a patient at risk of cardiac arrhythmia, the system comprising:
an ambulatory monitor device configured to:
sense a cardiac signal from the patient via a cardiac sensor; and
intermittently collect data segments of the sensed cardiac signal in accordance with a data segment collection rate or schedule over a monitoring period, the data segments each having a segment duration; and
an arrhythmia analysis device communicatively coupled to the ambulatory monitor device, the arrhythmia analysis device configured to detect a cardiac arrhythmia using the intermittently collected data segments received from the ambulatory monitor device,
wherein the ambulatory monitor device is configured to transmit the intermittently collected data segments to the arrhythmia analysis device in accordance with a transmission schedule or in response to a trigger event.

2. The system of claim 1, wherein:
the arrhythmia analysis device is configured to determine a confidence of the detection of the cardiac arrhythmia; and
the ambulatory monitor device is configured to adjust the segment duration, including to increase the segment duration in response to the determined confidence falling below a confidence threshold, and to maintain or decrease the segment duration in response to the determined confidence exceeding a confidence threshold.

3. The system of claim 1, wherein:
the arrhythmia analysis device is configured to determine an instability of the detected cardiac arrhythmia; and
the ambulatory monitor device is configured to adjust the segment duration, including to increase the segment duration in response to the determined instability exceeding an instability threshold, and to maintain or decrease the segment duration in response to the determined instability falling below the instability threshold.

4. The system of claim 1, wherein the ambulatory monitor device is configured to collect the data segments periodically with a data collection period longer than the segment duration.

5. The system of claim 1, wherein the ambulatory monitor device is configured to intermittently collect the data segments in accordance with a data collection schedule such that a time interval between at least two adjacent collections is longer than the segment duration.

6. The system of claim 1, wherein the ambulatory monitor device is configured to adjust the data segment collection rate or schedule, including to:
intermittently collect the data segments in accordance with a first data segment collection rate or schedule over a first monitoring period; and
intermittently collect the data segments in accordance with a second data segment collection rate or schedule different than the first data segment collection rate or schedule over a second monitoring period.

7. The system of claim 6, wherein the arrhythmia analysis device is configured to determine a characteristic of the detected cardiac arrhythmia,
wherein the ambulatory monitor device is configured to adjust the data segment collection rate or schedule based on the determined characteristic of the detected cardiac arrhythmia.

8. The system of claim 7, wherein the characteristic of the detected cardiac arrhythmia includes one or more of a heart rate or a duration of the detected cardiac arrhythmia.

9. The system of claim 6, wherein:
the arrhythmia analysis device is configured to determine a risk of the cardiac arrhythmia in the patient; and
the ambulatory monitor device is configured to adjust the data segment collection rate or schedule based on the risk of the cardiac arrhythmia.

10. The system of claim 6, wherein the arrhythmia analysis device is configured to determine a risk of the cardiac arrhythmia in the patient based on at least one of a physiological signal different from the cardiac signal sensed from the patient, a time of a day during the monitoring period, or an arrhythmia history of the patient.

11. The system of claim 1, wherein the arrhythmia analysis device is configured to detect the cardiac arrhythmia including an atrial fibrillation (AF) episode from each of the intermittently collected data segments.

12. The system of claim 11, comprising a user interface coupled to the arrhythmia analysis device, the user interface is configured to receive a user adjudication of the detected cardiac arrhythmia as a true-positive or a false-positive detection,
wherein the ambulatory monitor device is configured to adjust one or more of the segment duration or the data segment collection rate or schedule in response to the true-positive detection.

13. A method of monitoring a patient at risk of cardiac arrhythmia, the method comprising:
sensing a cardiac signal from the patient using a cardiac sensor;
intermittently collecting data segments of the sensed cardiac signal in accordance with a data segment collection rate or schedule over a monitoring period using an ambulatory monitor device, the data segments each having a segment duration;
transmitting the intermittently collected data segments to an arrhythmia analysis device in accordance with a transmission schedule or in response to a trigger event; and
detecting a cardiac arrhythmia using the intermittently collected data segments received from the ambulatory monitor device using the arrhythmia analysis device.

14. The method of claim 13, comprising:
determining a confidence of the detection of the cardiac arrhythmia; and
adjusting the segment duration, including increasing the segment duration in response to the determined confidence falling below a confidence threshold, and maintaining or decreasing the segment duration in response to the determined confidence exceeding a confidence threshold.

15. The method of claim 13, comprising:
determining an instability of the detected cardiac arrhythmia; and
adjusting the segment duration, including increasing the segment duration in response to the determined instability exceeding an instability threshold, and maintaining or decreasing the segment duration in response to the determined instability falling below the instability threshold.

16. The method of claim 13, wherein intermittently collecting the data segments includes periodically collecting the data segments with a data collection period longer than the segment duration.

17. The method of claim 13, wherein intermittently collecting the data segments includes varying the data segment collection rate or schedule, such that the data segments are intermittently collected in accordance with a first data segment collection rate or schedule during a first monitoring period, and with a second data segment collection rate or schedule different than the first data segment collection rate or schedule during a second monitoring period different than the first monitoring period.

18. The method of claim 17, comprising determining a characteristic of the detected cardiac arrhythmia including one or more of a heart rate or a duration of the detected cardiac arrhythmia,
wherein varying the data segment collection rate or schedule is in accordance with the determined characteristic of the detected cardiac arrhythmia.

19. The method of claim 17, comprising receiving information about a risk of the cardiac arrhythmia in the patient,
wherein varying the data segment collection rate or schedule is in accordance with the risk of the cardiac arrhythmia.

20. The method of claim 13, comprising:
receiving a user adjudication including a true-positive or a false-positive designation of the detected cardiac arrhythmia; and
adjusting one or more of the segment duration or the data segment collection rate or schedule in response to a true-positive designation of the detected cardiac arrhythmia.
